(19) United States
(12) Patent Application Publication
(10) Pub. No.: US 2008/0279437 A1
(43) Pub. Date: Nov. 13, 2008

(54) SYSTEM AND METHOD FOR THE CENTRALIZED EDITING, PROCESSING, AND DELIVERY OF MEDICALLY OBTAINED OBSTETRICAL ULTRASOUND IMAGES

(76) Inventors: Dina M. Hendricks, Henderson, NV (US); Terri R. Truex, Henderson, NV (US)

Correspondence Address: KENEHAN & LAMBERTSEN, LTD;JOHN C LAMBERTSEN, 6900 WESTCLIFF DRIVE, SUITE 104, LAS VEGAS, NV, 89145-0195, US

(21) Appl. No.: 12/114,794

(22) Filed: May 4, 2008

Related U.S. Application Data
(60) Provisional application No. 60/915,881, filed on May 3, 2007.

(51) Int. Cl. A61B 8/13

(52) U.S. Cl. 382/131

(57) ABSTRACT

A system and method uses images and video acquired from a medically necessary obstetric ultrasound procedure. The images and video are processed at a centralized location to create an end product to provide to the obstetric patient as a keepsake. Processing the images at the centralized location enables sophisticated editing and packaging processes to be used that are neither cost effective nor time effective if implemented at each medical facility. The system and method processes images and video from multiple locations and creates an exemplary product from procedures that confirm to FDA standards.

### RELATED APPLICATIONS

The present application claims priority under 35 USC § 119(e) to U.S. Provisional Application No. 60/915,881 filed on May 3, 2007, which is incorporated by reference herein.

### BACKGROUND OF THE INVENTION

1. Field of the Invention

The present invention is generally in the field of ultrasound (ultrasonic) imaging, and, more particularly, is in the field of acquiring, editing, processing and delivery of ultrasound images obtained during a medical ultrasound session for an obstetrical patient.

2. Description of the Related Art

Ultrasound imaging provides a noninvasive method for obtaining in utero images of a developing fetus. Such imaging may be used, for example, to provide information to a medical professional to assist in evaluating the condition of the developing fetus. Although considered to be a safe procedure when used as part of a medically necessary procedure, the US Food and Drug Administration and various medical authorities do not approve of the use of obstetrical ultrasound for non-medical use, such as for the sole purpose of obtaining “keepsake” images of the fetus. Furthermore, many medical professionals do not consider providing images to their patients as part of their medical practice. Accordingly, in many cases, if the medical professional provides any image to the expectant patient at all, the image probably cannot be considered to be keepsake quality and may not provide any sentimental value to the expectant patient and her family and friends.

### SUMMARY OF THE INVENTION

One aspect in accordance with the embodiments disclosed herein is a system and method that provides a consumer (e.g., an expectant mother) with a product that uses images obtained during a medically necessary ultrasound procedure. Although the images are produced during a medical procedure rather than a non-medical procedure, the images provide a level of quality and service that are not offered by medical providers.

In accordance with the system and method described and claimed herein, all materials required to produce the images for the consumer are provided to the medical provider by the image producer, which is identified by the trade name “First Memories.” The materials include marketing materials that the medical provider makes available to the obstetric patients. The marketing materials include an offer to the obstetric patients to purchase the First Memories product. The medical provider only has to record the ultrasound procedure in accordance with criteria established by First Memories. In particular, the medical provider is asked to only record ultrasound examinations of patients with fetuses of at least 14 weeks gestational age and only when the medical provider expects that the medically prescribed ultrasound examination will be at least 5 minutes in duration. The criteria are consistent with acceptable medical practice with respect to the ultrasonic procedure. The medical provider has the sole discretion to offer the First Memories product at the conclusion of the obstetric examination. If the patient elects to purchase the First Memories product, medical provider gives the patient an order form that includes package selection, shipping, and billing information. The order form also includes a disclosure and release form that must be checked and signed by the patient. The disclosure and release form explicitly releases the images to First Memories so that the medical provider is authorized to release the images, which may be considered to be confidential medical information. The disclosure and release form also expressly releases the physician from liability with regard to the images obtained. The disclosure and release form also expressly states that the images created by First Memories are for viewing purposes only and are not to be used for further medical evaluation.

The image producer, First Memories, also provides all necessary shipping envelopes to the medical provider to enable the medical provider to send a DVD with video and a CD with still images to First Memories for processing. First Memories edits the video and the still images to create a multi-function DVD that includes an edited video with background music, a picture slideshow with background music, and an advertiser section. First Memories also prints images onto photo paper and creates an album with images. All products produced by First Memories are labeled as First Memories products, and the DVD, the CD, and the photo album are shipped from the First Memories office directly to the customer within approximately seven business days after the time of the obstetric ultrasound procedure. The labeling and shipping of the First Memories products relieves the medical provider from any further involvement in the generation of the products and also serves as a reminder to the recipient that the products received are keepsake products and are not to be considered as medical images.

First Memories also maintains a website that lists medical providers that cooperate with First Memories. The website also lists package information and enables a patient of one of the cooperating medical provides to order image packages online. The website also includes a private section that is accessible only by authorized medical providers. The private section provides details regarding the marketing of the First Memories product and explains the payment procedure.

### BRIEF DESCRIPTION OF THE DRAWINGS

The foregoing aspects and other aspects of this disclosure are described in detail below in connection with the accompanying drawing figures in which:

FIG. 1 illustrates a simplified diagram of the components of the system used to acquire the images in accordance with a preferred embodiment;

FIG. 2 illustrates a scanned representation of an exemplary order form that includes an exemplary disclosure and release form;

FIG. 3 illustrates a simplified flow diagram of the processing of the DVD and CD that arrive at the centralized processing facility;

FIG. 4 illustrates a flow chart of the process that assembles the multifunction DVD product; and

FIG. 5 illustrates a flow chart of the process that assembles the photo album product.

### DETAILED DESCRIPTION OF A PREFERRED EMBODIMENT

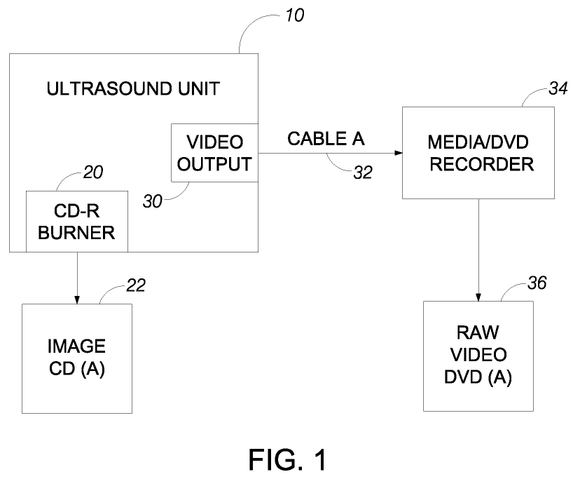

FIG. 1 illustrates a simplified diagram of the components of the system used to acquire the ultrasound images that are used to produce keepsake images in accordance with a preferred embodiment. The system includes a conventional ultrasound unit 10. The ultrasound unit is used by a medical provider in a conventional, medically acceptable manner to perform a medically necessary ultrasound procedure on an expectant patient. Any such procedure is performed for the sole purpose of obtaining ultrasound images of a fetus in utero in accordance with acceptable medical practice and as prescribed by the patient's physician. The system and method described herein neither add to nor detract from the medically necessary ultrasound procedure. The processes performed by the system and method utilize the images produced by the medically necessary procedure to produce a product that provides the obstetric patient with lasting memories without subjecting the patient any unnecessary procedures.

Preferably, the medical provider only has to record the ultrasound procedure in accordance with criteria established by First Memories. In particular, the medical provider is asked to only record ultrasound examinations of patients with fetuses of at least 14 weeks gestational age and only when the medical provider expects that the medically prescribed ultrasound examination will be at least 5 minutes in duration. For example, if the medical provider is only performing an ultrasound examination having a short duration to check for the heart rate or to provide early dating of the development of a fetus (e.g., at 4-10 weeks), the images produced during such scans are not likely to be of sufficient number and quality to produce a desirable image package. The medical provider is not requested to extend an otherwise short examination in order to obtain additional images. Accordingly, the criteria are consistent with acceptable medical practice with respect to the ultrasound procedure. Furthermore, the medical provider has the sole discretion to offer the First Memories product at the conclusion of the obstetric examination.

As further shown in FIG. 1, the ultrasound unit 10 includes a conventional recorder (CD-R burner) 20 that stores still images that are created and stored within the ultrasound unit during the medically necessary procedure onto a recordable compact disk (CD-R). The decisions as to the still images to create and store are made by the medical provider in accordance with established medical criteria related to the examination of the patient. At the conclusion of the medically necessary procedure, the medical provider uses the CD-R burner to record a complete set of the images created during the procedure onto a specifically identified image CD-R 22 provided to medical provided by the image producer (First Memories) as an initial step in the process disclosed herein. In particular, the image CD-Rs are provided to the medical provider as part of the overall system and process of producing the keepsake images so that the medical provider has an adequate supply of the image CD-Rs at no cost to the medical provider. The medical provider may also use the CD-R burner to store some or all of the images from the procedure on a separate CD-R that the medical provider keeps as part of the patient's records related to the medically necessary procedure. The step of burning the images onto the CD-R for the image producer is advantageously performed during downtime between patients when the examination room is being prepared for the next patient. Accordingly, the short amount of time required to record the image CD-R does not affect the time required for the medically necessary procedure, and the step of producing the image CD—for the image producer is effectively transparent to the medical provider and to the patient.

As further shown in FIG. 1, the ultrasound unit 10 includes a video output interface 30 that provides digital video data that represents the motion of the fetus within the uterus. The image producer supplies a cable 32 (“cable A”) to the medical provider to interconnect the video output interface to a media/DVD recorder 34, which is also provided by the image producer. In the illustrated embodiment, the output interface provides S-video data, and the cable A is a conventional S-video cable that interconnects the S-video output interface of the ultrasound unit to an S-video input of the media/DVD recorder.

Preferably, the media/DVD recorder 34 is operational during the medically necessary procedure to record the real-time images of the fetus. The media/DVD recorder records the images onto a DVD (digital video disk or digital versatile disk) 36 that has sufficient storage to record the video data generated during the procedure. The video data recorded onto the DVD is raw video data that represents the images produced by the medically necessary procedure.

The media/DVD recorder 34 is configured so that the medical provider only has to load a DVD 36 into the recorder prior to the procedure, to start the recorder at the beginning of the procedure and to stop the recorder at the end of the procedure. In one embodiment, the DVDs are conventional blank DVDs, and the medical provider is instructed to format each DVD prior to use. The media/DVD recorder includes internal electronics and programming to perform the formatting in response to activation by the medical provider. Alternatively, the image producer provides a supply of properly formatted and appropriately identified DVDs to the medically provider so that the formatting step may be omitted.

At the conclusion of the procedure, when the examination room is being prepared for the next patient, the recorded DVD 36 is removed from the recorder 34 and an unrecorded DVD is inserted into the recorder. In preferred embodiments, the recorder may include label writing technology (e.g., LightScribe or other direct disc labeling technology) so that a label may be burned onto the DVD to identify the date and time of the procedure that produced the data recorded on the DVD.

At a selected time before, after or during the medically necessary procedure, the medical provider or office personnel associated with the medical provider inform the obstetric patient of the availability of the keepsake quality images produced by the image producer (First Memories). If the patient expresses an interest in the keepsake quality images, the patient is provided additional information and forms to complete that initiate the ordering process and that enable the medical provider to release the images to the image producer. As discussed above, the medically necessary procedure is not altered if the patient chooses to purchase the keepsake quality images. Accordingly, the patient may be informed of the availability of images at any time without affecting either the procedure of the availability of the images.

The medical provider may even allow the patient to defer a decision to purchase the keepsake quality images for a reasonable time after the procedure is completed if the medical provider has room to store and is willing to store the image CD-R 22 and the video DVD 36 until the patient decides. For example, if a patient is scheduled for follow-up ultrasound examinations as the fetus develops, the medical provider may store the CD-R and the video DVD without finalizing the discs so that additional information may be added to the discs during or at the conclusion of at least one subsequent examination. In the preferred embodiment, neither disc is finalized by the medical provider.

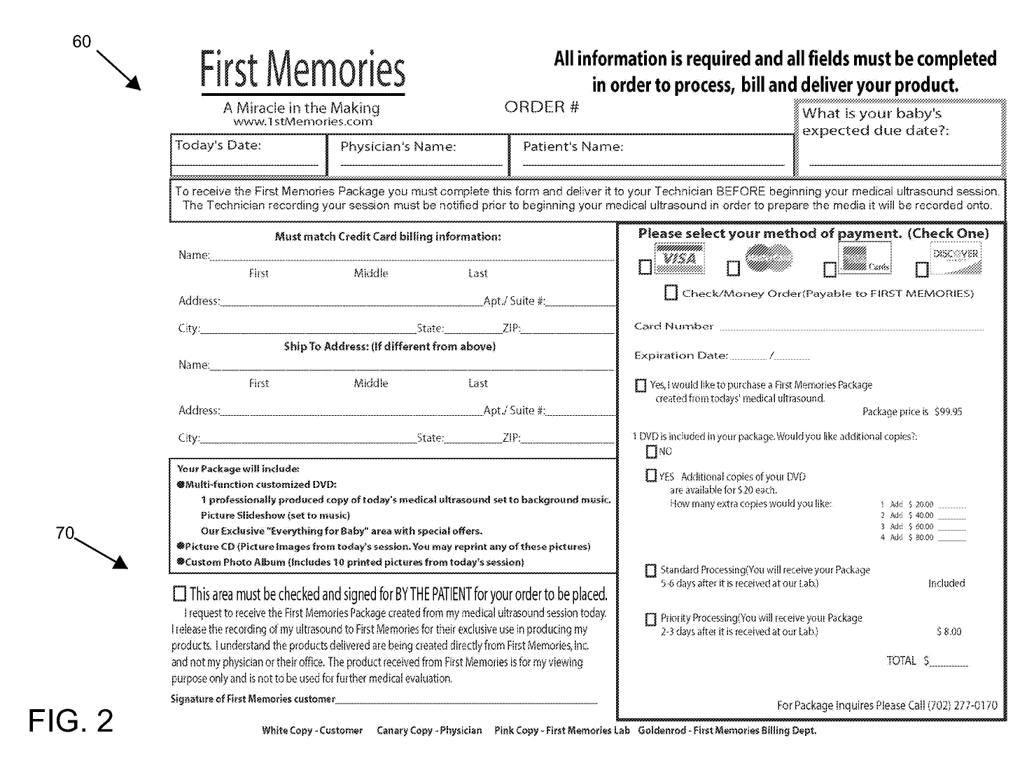

Preferably, an obstetric patient will choose to purchase the keepsake quality images at the conclusion of the examination procedure. If so, the patient completes and signs an order form, such as, for example, an exemplary order form 60 shown in FIG. 2. The patient uses the order form to select a desired image package and to provide payment information (e.g., credit card information or an enclosed check). The order form also includes address information for shipping the requested image package.

The patient also completes and signs a disclosure and release form, such as, for example, an exemplary disclosure and release form 70. As shown in FIG. 2, in the illustrated embodiment, the disclosure and release form 70 is included as part of the order form 60 (e.g., in the lower left corner of the form 60). In the disclosure and release form, the patient explicitly authorizes the medical provider to send the images on the CD-R 22 and the video data on the DVD 36 to the image producer (First Memories). The disclosure and release form further requires the patient to acknowledge that the images to be produced are not to be used for medical purposes.

After the patient completes the order form 60 and the disclosure and release form 70, the two forms, an optional check for payment, the CD-R 22 and the video DVD 36 are placed in a special, preaddressed and postage paid mailing envelope to send the discs and the information to the image producer (First Memories). The medical provider uses the envelope to send the DVD, the CD and the completed forms to a centralized processing facility of the image producer. Note that the medical provider mails the envelope rather than relying on the patient to mail the envelope with the sensitive patient information. When the proper procedures are followed, the patient information is transferred from the control of the medical provider to the control of the image producer to avoid accidental disclosure of the information.

When the envelope with the completed order form 60, including the signed disclosure and release form 70, the image CD-R 22, and the video DVD 36 arrive at the centralized processing facility, the contents of the envelope are removed and reviewed to confirm that that the forms are properly completed and that the payment information (e.g., credit card information) or payment (e.g., a check) is included. Optionally, the patient may have completed an order via the image producer's website. If the order form indicates that the order was completed or was to be completed via the website, the information in the envelope is matched with information received via the website to verify that payment was received and that the image CD-R and the video DVD should be processed to produce the requested keepsake image package.

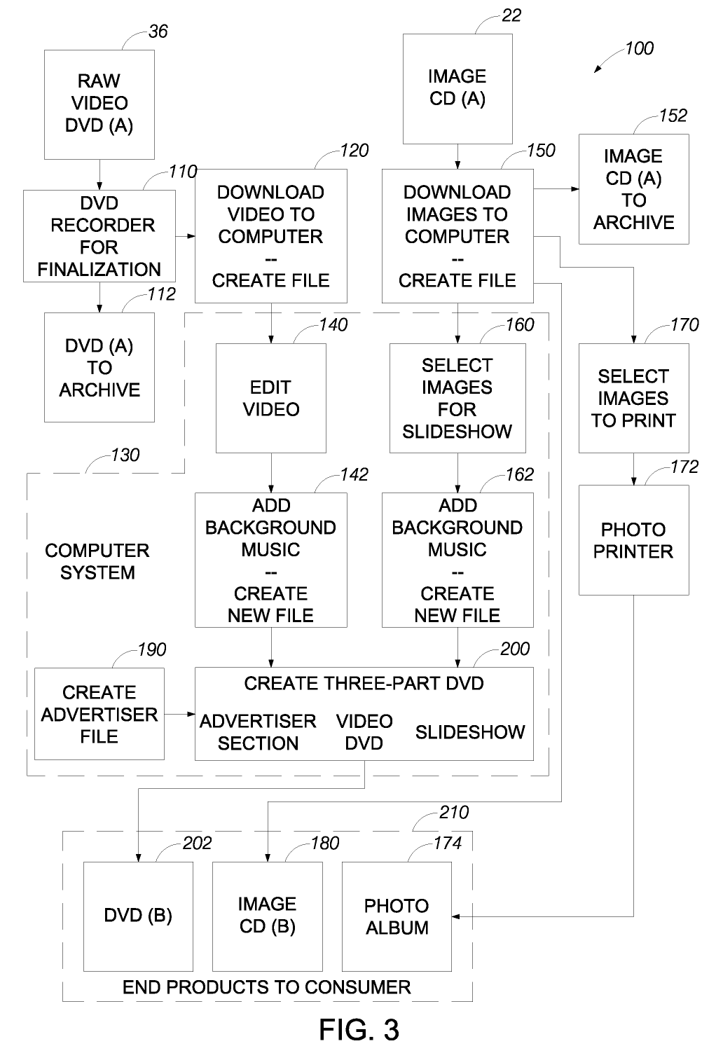

FIG. 3 illustrates a simplified flow diagram 100 of the processing of the video DVD (raw video DVD (A)) 36 and the image CD-R (image CD (A)) 22 that arrive at the centralized processing facility.

The video DVD 36 is first inserted into a DVD recorder 110 wherein the DVD is finalized such that additional data can no longer be written onto the media of the DVD. The finalized DVD is thus transformed into an archive DVD (A) and is stored in an archive location as represented by an archive function 112. The data on the finalized DVD is also transferred from the DVD recorder via a download video process 120 to a computer system 130 (represented by a dashed outline). The computer system advantageously comprises desktop or workstation computer such as an IBM-compatible or an Apple Mac-compatible system that includes conventional high-end image processing software and high-end sound processing software. Such software is available from a number of sources, and the operational characteristics of such software are well known to persons in the image processing and sound processing arts. Preferably, the computer system includes enhanced video processing and sound processing hardware to increase the performance of the computer system.

The image processing software in the computer system 130 is represented by an edit video function 140. The image producer uses the edit video function to manipulate the video images to produce one or more sequences of frames that are pleasing to watch and that show the movement of the fetus within the uterus. The sequences of video frames are synchronized with background music in an add background music function 142. It should be understood that the add background music function and the edit video function are advantageously components of the same processing software in many systems. It should be further understood that the video editing and the music editing are iterative processes that are repeated until the artist controlling the computer system has created a pleasing product suitable to be sent to the obstetric patient as a portion of the keepsake quality image package. After the artist is satisfied with the quality of the video and music, the data representing the video and music are stored in a new file.

The image CD-R 22 received from the medical provider is processed in a similar manner to the video DVD 36. The image CD-R is inserted into a CD reader and the digitized images are transferred into the computer system 130 via a download images process 150. Alternatively, the images may be transferred into a second computer system (not shown) so that the images may be processed by another artist in parallel with the processing of the video data. After transferring the image data to the computer system, the image CD-R is transferred to archival storage as represented by an archive function 152

Within the computer system 130, image processing software, represented by a select images for slideshow procedure 160, is used to select images for a slideshow and organize the images into a sequence of images in a pleasing arrangement determined by the artist. The image processing software may be part of the same software that processes the video images or the image processing software may be optimized for processing still images.

After selecting the images and the sequence in which the images are presented as a slideshow, the artist adds background music to the slideshow and synchronizes the transitions between images to the music. After the artist is satisfied with the quality of the images in the slideshow and the music accompanying the slideshow, the data representing the slideshow and the music are stored in a new file.

As further shown in FIG. 3, the image data on the image CD-R 22 is reviewed and images are selected having particular artistic value in a select images process 170. The selected images are printed in a high quality photo printer 172 and are assembled in a photo album 174 so that the selected images can be viewed without requiring a computer, a video system or other electronic equipment.

As further shown in FIG. 3, the downloaded still images from the image CD-R 22 are output directly to a CD-R recorder (not shown) to create a second copy 180 of the image CD-R (identified as image CD (B)).

As further shown in FIG. 3, the computer system 130 further includes a advertiser creation process 190 that creates an advertiser data file. The advertiser file includes, for example, information for ordering additional copies of the keepsake images for relatives and friends. The advertiser file may also include images and other information regarding other products available from the image producer that may interest the obstetric patient. The advertiser file may also advantageously include paid advertisements from other vendors who desire to market products to families with infants and young children.

As further shown in FIG. 3, the file for the edited video from the process 142, the file with the slideshow from the process 162 and the advertiser file from the process 190 are combined in a process 200 to create a three-part DVD 202 (identified as DVD (B)). In particular, the three-part DVD 202 includes the advertiser section, the video DVD section and the image slideshow section that the consumer (e.g., the obstetric patient) may selectively access via a conventional DVD player in a computer system, in a home video system, a portable video system or other equipment capable of reading and displaying data from a DVD.

As further shown in FIG. 3, the DVD (B) 202, the image CD (B) 180 and the photo album 174 are packaged as a combined end product 210 to ship to the consumer.

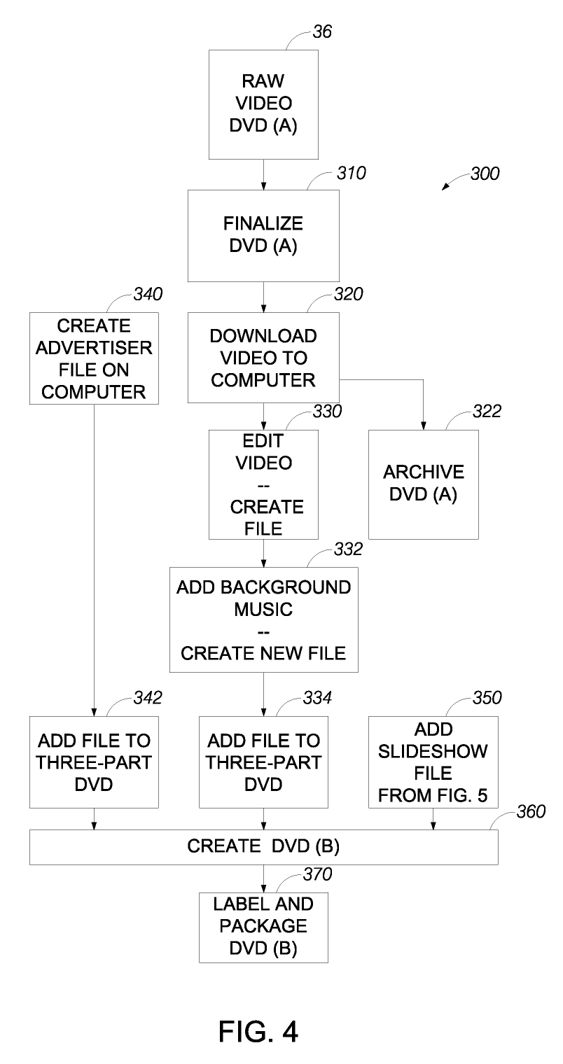

FIG. 4 illustrates a flow chart 300 that focuses on the portions of the process 100 that assembles the multifunction DVD product 202. As described above with respect to FIG. 3, the process starts with the raw video data on the raw video DVD (A) 36. The DVD is finalized in a process 310 corresponding to the operation of the DVD recorder 110 in FIG. 3. After the DVD is finalized, a process 320 downloads the raw video data to the computer. After the raw video data are downloaded to the computer, the finalized DVD is archived in a process 322.

The downloaded video data in the computer is edited in a video editing process 330 to create an edited video file. Then a background music process 332 adds background music to the edited video file to create a new file the combines the video and audio. It should be understood that the video editing process and the background music process may be iterative such that the video file may be further edited to conform to the background music added during the background music process. After completing the video editing and background music processes, the new file created by the processes is added to a three-part DVD file in a process 334. The three-part DVD file becomes part of the three-part DVD (B) 202 described above with respect to FIG. 3.

As further shown in FIG. 4, the flow chart 300 further includes a create advertiser file process 340 that creates an advertiser file as discussed above in connection with FIG. 3. The advertiser file created in the advertiser file process is added to the three-part DVD file in process 342.

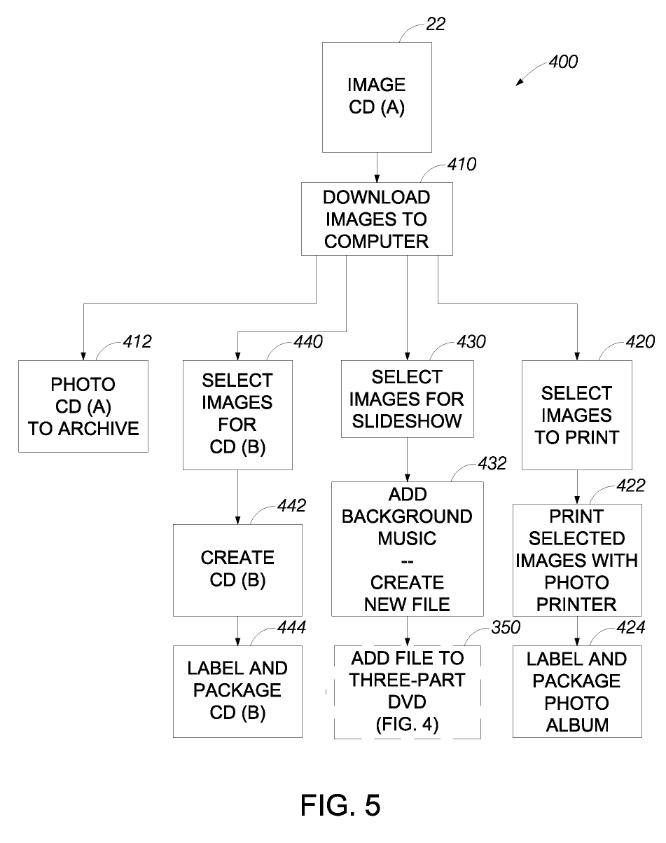

As further shown in FIG. 4, a slideshow file is added to the three-part DVD file in a process 350. The process that generates the slideshow file is described above with respect to the process flow diagram of FIG. 3 and is described below with respect to the flow chart of FIG. 5.

As further shown in FIG. 4, the advertiser file added by the process 342, the video file added by the process 334 and the slideshow file added by the process 350 are transferred to a DVD in a process 360 to create the three-part DVD (B) 202 described above. The completed DVD (B) is labeled and packaged in a process 370. The labeled and packaged DVD (B) is included in the end product 210 shipped to the customer as described above with respect to FIG. 3.

FIG. 5 illustrates a flow chart 400 that focuses on the portions of the process 100 of FIG. 3 that processes the image CD-R (image CD (A)) 22 from the medical provider and that creates the consumer's copy of the image CD (B) 180, the photo album 174 and the slideshow portion of the three-part DVD (B) 202. As illustrated, the image data on the image CD (A) is downloaded to the computer in a process 410. After downloading the image data, the original image CD (A) is archived in a process 412.

As further shown in FIG. 5, images in the downloaded image data are selected in a process 420 to identify a set of images to print. Then, in a process 422, the selected images are printed in a photo printer to create a set of printed images. In a process 424, the printed images are labeled and are packaged in the photo album 174 to include in the end product 210 to ship to the consumer.

As further shown in FIG. 5, images in the downloaded image data are selected in a process 430 to identify a set of images to include in a slideshow. The selected images are arranged for presentation in a selected sequence. Then, in a process 432, background music is added to the slideshow images and the music and the slideshow sequencing are synchronized. The synchronized images and background music are saved in a new file. The new file is transferred to the process 350 in FIG. 4 to become the slideshow portion of the three-part DVD 202. The process 350 is reproduced in dashed lines in FIG. 5 to clarify that the process is the process illustrated in FIG. 4.

As further shown in FIG. 5, the images in the downloaded image data are selected in a process 440 to be used to create the consumer image CD (B) 180. The image selection process may select all of the images in the downloaded data; however, preferably, images are selected for clarity and are also selected to avoid multiple images that are substantially similar. Then, in a process 442, the selected images are transferred to the image CD (B). In a process 444, the image CD (B) is labeled and packaged to be included in the end product 210 (see FIG. 3) to be shipped to the consumer.

One skilled in art will appreciate that the foregoing embodiments are illustrative of the present invention. The present invention can be advantageously incorporated into alternative embodiments while remaining within the spirit and scope of the present invention, as defined by the appended claims.

What is claimed is:

1. A system that converts ultrasound images of a fetus generated by an ultrasound unit operated by a medical provider during a medically necessary obstetric examination procedure into an audio visual product suitable for showing to non-medical personnel, the system comprising:
a compact disk recorder that records still images produced by the ultrasound unit onto compact disks (CDs) during the course of the medically necessary obstetric examination procedure;
a digital versatile disk (DVD) recorder coupled to the ultrasound unit to record video data generated by the ultrasound unit during the course of the medically necessary obstetric examination procedure onto digital versatile disks (DVDs);
a computer system at a centralized processing facility that receives the still images and the video data, the computer system including:
a first process that edits the still images produced by the medically necessary obstetric examination procedure to convert selected still images into a slideshow synchronized with background music;
a second process that edits the video data produced by the medically necessary obstetric examination procedure to produce an edited version of the video data synchronized with background music and suitable to be shown to non-medical personnel; and
a third process that combines the still images produced by the first process and the video data produced by the second process onto a single DVD.

2. The system as defined in claim 1, further comprising a process that adds advertising data to the DVD produced by the third process.

3. The system as defined in claim 1, further comprising a process that prints selected still images as photographs to include in a photo album.

4. The system as defined in claim 1, wherein the still images and the video data are produced without altering the medically necessary obstetric examination procedure.

5. A method for converting ultrasound images of a fetus generated by an ultrasound unit operated by a medical provider during a medically necessary obstetric examination procedure into an audio visual product suitable for showing to non-medical personnel, the method comprising:
recording still images produced by the ultrasound unit onto compact disks (CDs) during the course of the medically necessary obstetric examination procedure;
recording video data generated by the ultrasound unit during the course of the medically necessary obstetric examination procedure onto digital versatile disks (DVDs);
receiving the still images and the video data at a centralized processing facility;
editing the still images produced by the medically necessary obstetric examination procedure to convert selected still images into a slideshow synchronized with background music;
editing the video data produced by the medically necessary obstetric examination procedure to produce an edited version of the video data synchronized with background music and suitable to be shown to non-medical personnel; and
combining the slideshow of the still images and the edited version of the video data onto a single DVD.

6. The method as defined in claim 5, further comprising adding advertising data to the single DVD.

7. The method as defined in claim 5, further comprising printing selected still images as photographs to include in a photo album.

8. The method as defined in claim 5, wherein the still images and the video data are produced by the medical provider using the ultrasound unit without altering the medically necessary obstetric examination procedure.

9. A method for converting ultrasound images of a fetus generated by an ultrasound unit operated by a medical provider during a medically necessary obstetric examination procedure into an audio visual product suitable for showing to non-medical personnel, the method comprising:
recording video data generated by the ultrasound unit during the course of the medically necessary obstetric examination procedure onto digital versatile disks (DVDs);
receiving the video data at a centralized processing facility;
editing the video data produced by the medically necessary obstetric examination procedure to produce an edited version of the video data synchronized with background music and suitable to be shown to non-medical personnel; and
recording the edited version of the video data onto a single DVD.
